(19) United States
(12) Patent Application Publication
(10) Pub. No.: US 2026/0130960 A1
(43) Pub. Date: May 14, 2026

(54) SYNERGISTIC HERBAL COMPOSITION WITH NOOTROPIC ACTIVITY FOR IMPROVING COGNITIVE FUNCTION AND GAMING PERFORMANCE

(71) Applicant: LAILA NUTRA PRIVATE LIMITED, Vijayawada, Andhra Pradesh (IN)

(72) Inventors: Ganga Raju GOKARAJU, Vijayawada, Andhra Pradesh (IN); Rama Raju GOKARAJU, Vijayawada, Andhra Pradesh (IN); Kiran BHUPATHIRAJU, Vijayawada, Andhra Pradesh (IN); Venkateswarlu SOMEPALLI, Vijayawada, Andhra Pradesh (IN); Venkata Kanaka Ranga Raju GOKARAJU, Vijayawada, Andhra Pradesh (IN); Venkata Krishna Raju ALLURI, Vijayawada, Andhra Pradesh (IN); Krishanu SENGUPTHA, Vijayawada, Andhra Pradesh (IN); Trimurtulu GOLAKOTI, Vijayawada, Andhra Pradesh (IN)

(21) Appl. No.: 19/120,492

(22) PCT Filed: Oct. 17, 2023
(86) PCT No.: PCT/IN2023/050949

§ 371 Date: Apr. 11, 2025

(30) Foreign Application Priority Data
Oct. 31, 2022 (IN) 202241061917

(51) Int. Cl. A61K 36/23; A61K 33/08; A61K 33/10; A61K 36/185; A61K 45/06; A61P 25/28

(52) U.S. Cl. CPC A61K 36/23; A61K 33/08; A61K 33/10; A61K 36/5777; A61K 45/06; A61P 25/28; A61K 2236/33; A61K 2236/331; A61K 2236/333; A61K 2236/51; A61K 2236/53

(57) ABSTRACT

The present invention discloses synergistic herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; process for their preparation, methods of treatment and use of such nootropic compositions for improving cognitive functions or mental abilities and gaming performance.

### TECHNICAL FIELD OF THE INVENTION

The present invention relates to synergistic herbal compositions comprising combination of; a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; for increasing nootropic activities that are essential for improving cognitive functions and boosting gaming performance.

### BACKGROUND OF THE INVENTION

Cognition is defined as “the brain's ability to acquire, process, store, and retrieve information”. It involves several critical elements, such as attention, perception, memory, motor skills, executive functioning, and verbal/language skills. Age-associated cognitive decline has been well documented in the scientific literature. Oxidative stress and inflammation have been implicated in aging and age-associated cognitive decline in neurodegenerative disorders, including Alzheimer's disease (AD). Acetylcholine (ACh), the first neurotransmitter identified, utilized by all cholinergic neurons, has a vital role in the peripheral and s central nervous system (CNS). The cholinergic system plays a crucial role in higher cognitive functions, specifically in learning and decision-making. Ach is involved in neuromodulation through the differential activation of the nicotinic and muscarinic receptors. Several studies have demonstrated that ACh is important for memory and for modulation of acquisition, encoding, focus, concentration, consolidation, and reconsolidation. Ach is rapidly inactivated by the enzyme Acetyl cholinesterase (AChE).

In recent years, electronic sports (esports) or video game playing has become a very popular recreational and professional activity globally, particularly among younger individuals. eSports require task switching, quick reactions to multiple auditory and visual cues, decision-making, executive function, memory, spatial awareness, fine motor skills, and mood improvement. eSports is emerging as an area of research focusing on cognitive processes that help executive functions and their impact on performance in competitive game play. As the participants in esports competitions/tournaments often play for hours per session over a series of days, maintaining cognitive and executive function, concentration, and fine motor skills is of paramount importance. For this reason, many video gainers rely on chemicals such as caffeine or caffeinated beverages to improve cognitive functions, particularly executive function, memory, creativity, motivation, alertness, and delay fatigue. However, excessive caffeine intake may promote nervousness and/or interfere with fine motor skills or have no effect on e-sport performance. It can further result in side effects such as caffeine crash, caffeine-induced irritability, etc. Hence, there is a growing interest in developing non-caffeine herbal extracts or botanical compositions for improving gaming performance and mood without the adverse effects commonly associated with caffeine or caffeinated products.

Indian patent publication No. IN304584 discloses a synergistic pharmaceutical composition for serotonin reuptake inhibition, wherein said composition comprises pentacyclic terpenoid glycosides, preferably asiaticoside and madecassoside optionally, along with excipients.

Another Indian patent publication No. 1633/MUM/2011 discloses a method of managing disease conditions selected from a group comprising Hypercortisolemia, Headache disorder, and Neuropathic pain or any combination of conditions thereof, said method comprising an act of administering pharmaceutically effective amount of a composition comprising asiaticoside and madecassoside, optionally along with at least one excipient, to a subject in need thereof.

Other Indian patent publication No. 202011020939 discloses a herbal composition for relieving Obsessive-Compulsive Disorder and general mental illness, said herbal composition comprising a plurality of herbs including a whole plant of Centella asiatica; a whole plant of Convolvulus pluricaulis; a root from Acorus calamus; a root from Glycyrrhiza glabra; a stem from Tinospora cordifolia; a tuber and root from Asparagus racemosus; a root from Nardostachys jatamansi; a root from Saussurea lappa; a bark from Betula utilis; a seed from Celastrus paniculatus; a root from Withania somnifera; and a whole plant of Ocimum tenuiflorum; wherein each of said plurality of herbs being in a range of 4-20% w/w of said herbal composition, and wherein said herbal composition is administered to a subject in the form of a herbal smoke on combustion of said herbal composition.

Other Indian patent publication No. 1167/CHF/2009 discloses a medicinal or herbal composition comprising a mixture of Acorns calamus in an amount of about 30% by weight of the composition; Clytoria ternalea in an amount of about 30% by weight of the composition; Glycyrrhiza glabra in an amount of about 10% by weight of the composition; Eleocarpus ganiatrus in an amount of about 10% by weight of the composition; Bacopa monnieri in an amount of about 10% by weight of the composition; and Centella asiatica in an amount of about 10% by weight of the composition.

Another Indian patent publication No. IN236752 discloses a herbal formulation(s) comprising the extracts of the pharmacologically effective form obtained from Tinospora cordifolia, Centella asiatica, Withania somnifera, Mucuna pruriens. and Curcuma longa in pharmaceutically acceptable dosage optionally along with an additive useful as brain tonic and in treatment of senile and presenile dementia.

Other Indian patent publication No. IN238309 discloses a synergistic herbal composition as a brain tonic, cognition, recalling of thoughts, and as an anti-oxidant capable of treating or preventing amnesia and having properties for improving memory; said composition comprising pharmaceutically acceptable amounts of extracts from plants Centella asiatica in the range of 1 to 15% and Sesamum indicum in the range of 2 to 20% optionally along with pharmaceutically acceptable salt/s, carrier/s or diluent/s such as herein described.

Chinese Patent Publication No. CN102512431 discloses the application of a Centella asiatica triterpenoid component to preparing an anti-craniocerebral injury product, namely a new application of asiaticoside and aglycone asiatic acid thereof, madecassoside and aglycone madecassoside thereof to the preparation of medicaments for treating craniocerebral trauma and sequelae thereof.

Another Chinese Patent Publication No. CN102389437 discloses the application of Centella triterpenes in preparing products for improving cerebral apoplexy sequelae, in particular to the new use of Centella asiatica alpha-amyrin triterpenes (including asiaticoside, asiatic acid, hydroxyl asiaticoside, and madecassic acid) in preparing products for improving cerebral apoplexy sequelae.

Korean Patent Publication No. KR960022436 discloses an asiatic acid used as an agent for curing trauma, which is produced by using a madecassic acid obtained from Centella asiatica, as a starting material.

However, the prior art fails to provide a precise and effective composition for increasing nootropic activities that are essential for improving cognitive functions and gaming performance.

### OBJECT OF THE INVENTION

Therefore, the primary object of the present invention is to provide synergistic and safe herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; process for their preparation and method of treatment for increasing nootropic activities that are essential for improving cognitive functions and boosting gaming performance.

### SUMMARY OF THE INVENTION

Accordingly, in one aspect, the present invention provides synergistic herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; for increasing nootropic activities that are essential for improving cognitive functions and boosting gaming performance.

In another aspect, the present disclosure provides synergistic herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; and further containing optionally at least one component selected from pharmaceutically or nutraceutically or dietically acceptable excipients, carriers, and diluents.

In another aspect, the present disclosure provides a process for the preparation of compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; and further containing optionally at least one component selected from pharmaceutically or nutraceutically or dietically acceptable excipients, carriers, and diluents.

In a further aspect, the present disclosure provides method of improving at least one benefit related to cognitive function, mental ability and/or nootropic activity selected from alertness, vigilance, attention, focus/concentration, neuromotor function (reaction time), memory (verbal, working, visual, logical), intelligence, learning, acquisition, retention, recall information/communication, perception, reasoning, problem solving, decision making, and; reducing mental fatigue and improving gaming performance; wherein the method comprises supplementing the human in need thereof with an effective dose of a composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents.

In a further aspect, the present disclosure provides use of the present herbal composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; and further containing optionally at least one component selected from pharmaceutically or nutraceutically or dietically acceptable excipients, carriers, and diluents; for improving at least one benefit related to cognitive function, mental ability and/or nootropic activity selected from alertness, vigilance, attention, focus/concentration, neuromotor function (reaction time), memory (verbal, working, visual, logical), intelligence, learning, acquisition, retention, recall information/communication, perception, reasoning, problem solving, decision making, and; reducing mental fatigue and improving gaming performance.

### DETAILED DESCRIPTION OF THE INVENTION

The invention will now be described in detail in connection with certain preferred and optional embodiments so that various aspects thereof may be more fully understood and appreciated.

Unless specified otherwise, all technical and scientific terms herein have the same meaning as commonly understood by one of ordinary skill in the art to which this invention belongs. To describe the invention, certain terms are defined herein specifically as follows.

The terms “metal salt”, “metal complex” and “metal chelate” convey the same meaning and are used interchangeably in the specification. Thus, Centella asiatica extract containing magnesium salt of asiatic acid and magnesium salt of madecassic acid; Centella asiatica extract containing magnesium complex of asiatic acid and magnesium complex of madecassic acid; Centella asiatica extract containing magnesium chelate of asiatic acid and magnesium chelate of madecassic acid; convey the same meaning and are interchangeably used throughout in the specification. The terms “centellosides” or “total asiaticosides” comprise asiaticoside, madecassoside, asiatic acid, and madecassic acid; they convey the same meaning and are interchangeably used throughout in the specification.

Unless stated to the contrary, any of the words, “including,” “includes,” “comprising,” and “comprises” mean “including without limitation” and shall not be construed to limit any general statement that it follows to the specific or similar items. The terms “improve,” and “better,” as used herein, convey the same meaning and are interchangeable. The term “methods of obtaining health benefit” refers to inhibiting, preventing, or arresting the development of pathology (disease, disorder or condition) and/or causing the reduction, remission, or regression of a pathology. Those with skill in the art will understand that various methodologies and assays can be used to assess the development of pathology, and similarly, various methodologies and assays may be used to assess the reduction, remission, or regression of pathology.

The source of the herbs used in the invention is as follows:
1. Centella asiatica was collected from Aswaraopet village, Aswaraopet Mandal, Bhadradri Kothagudem district, Telangana state.
2. Theobroma cacao was collected from Kondalaraopalem village, Pedavegi Mandal, Eluru district, Andhra Pradesh.

Centella asiatica: It is a perennial and aromatic plant belonging to the Apiaceae family and is commonly known as Gotu Kola or Indian pennywort. It has been used as a medicine in the ancient Indian system to enhance memory and treat skin diseases and nerve disorders. In Chinese literature, it is reported as one of the “miracle elixirs of life” known over 2000 years ago. Besides its common use as a medicinal plant, it is also eaten fresh as salad, cooked as a vegetable, and blended as a drink. Centella asiatica is used for the treatment of a wide variety of disorders, such as skin diseases, rheumatism, inflammation, syphilis, mental illness, epilepsy, hysteria, asthma, dehydration, and diarrhea. The medicinal values of this plant are mainly attributed to the presence of triterpenes like asiatic acid, madecassic acid, asiaticoside, and madecassoside (FIG. 1). These four constituents are collectively represented as centellosides. Other constituents present in Centella asiatica include flavonoids such as quercetin, kaempferol and some phytosterols such as campesterol, sitosterol, and stigmasterol.

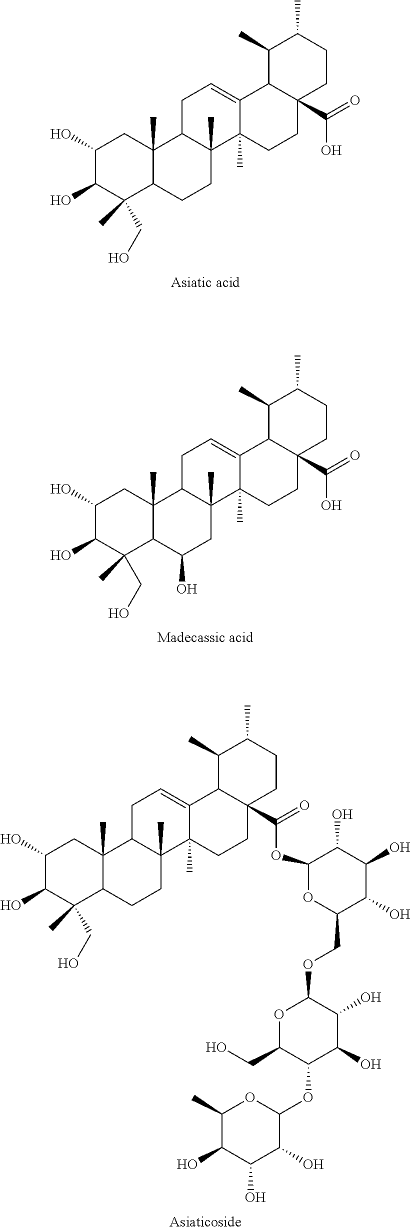

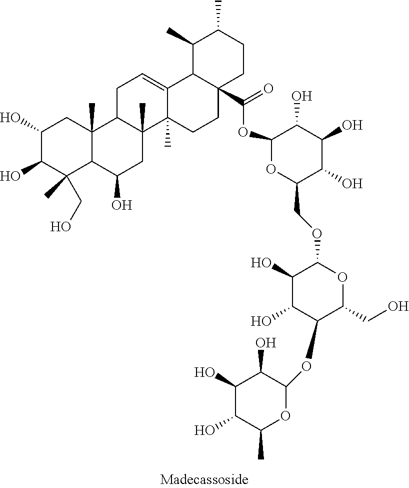

### FIG. 1: Chemical Structures of Asiatic Acid, Madecassic Acid, Asiaticoside and Madecassoside.

Many drug molecules containing organic acids are present in metal salt or metal complex form. It is the simplest way to modify the undesirable features of an organic acid. The typical suboptimal physicochemical or biopharmaceutical properties of an organic acid can be improved by pairing a metal ion to a salt form or complex of the parent compound.

Magnesium is one of the highly abundant essential minerals in the human body. Magnesium is a cofactor in more than 300 enzyme systems, including protein synthesis, muscle and nerve function, blood glucose control, and blood pressure regulation. Magnesium is required to synthesize DNA, RNA, and the antioxidant glutathione. It is essential in the manufacturing of adequate energy in the cells. It also plays a role in the active transport of calcium and potassium ions across cell membranes, which is important to nerve impulse conduction, neuromuscular coordination, muscle contraction, and normal cardiac function. Additionally, magnesium plays a prominent role in energy homeostasis and metabolism, and brain function. Adenosine triphosphate (ATP) is the energy currency of the body; but for its activity or to generate energy, ATP must be bound to a magnesium ion, generally to one of its phosphate groups.

The inventors presumed that Centella asiatica extract containing metal salt or complex or chelate of asiatic acid and madecassic acid along with asiaticoside and madecassoside would improve nootropic activities.

Thus, given the importance of magnesium, the inventors of the present invention prepared novel Centella asiatica extract, which inherently contains magnesium salt or complex of asiatic acid and magnesium salt or complex of madecassic acid along with asiaticoside and madecassoside.

Centella asiatica extract containing a magnesium salt or complex of asiatic acid and madecassic acid along with asiaticoside and madecassoside: Centella asiatica aerial parts were extracted with 70% aq. ethanol, and the extract was treated with magnesium oxide. The spent raw material was extracted with water, and the extract was treated with magnesium oxide. The blend of two extracts thus forms an extract comprising magnesium salt or complex of asiatic acid and magnesium salt or a complex of madecassic acid along with asiaticoside and madecassoside (CAMg-1). The extract thus obtained contains 13.03% of centellosides and 1.09% of magnesium (Example-1). For comparison, the blend of Centella asiatica 70% aq. ethanol extract and Centella asiatica spent water extract without magnesium was also prepared (Comparative example-1A). Similarly, Centella asiatica 70% aq. ethanol extract containing magnesium salt or complex of asiatic acid and magnesium salt or complex of madecassic acid along with asiaticoside and madecassoside (CAMg-2) was prepared. This extract contains 13.69% centellosides and 0.61% magnesium (Example-2). For comparison, Centella asiatica 70% aq. ethanol extract without magnesium salt or complex was also prepared (Comparative example 2A). Further, Centella asiatica water extract containing magnesium (CAMg-3), 70% aqueous methanol extract containing magnesium (CAMg-4), and 70% aqueous acetone extract containing magnesium (CAMg-5) were also prepared (Examples 3-5).

Salt or complex formation: The chemical structures of asiatic acid and madecassic acid contain a carboxylic acid group (see FIG. 1) capable of forming a metal salt or complex with a base. Centella asiatica extract, when treated with metal oxides, metal hydroxides, and metal carbonates etc., forms metal salt or complex of asiatic acid and madecassic acid. For example, the treatment of Centella asiatica extract with magnesium oxide followed by filtration of the solution to remove unreacted magnesium oxide gave Centella asiatica extract comprising magnesium salt or complex of asiatic acid and magnesium salt or complex of madecassic acid along with asiaticoside and madecassoside. The presence of magnesium (1.09%) in ICP-MS confirms the formation of magnesium salt. As magnesium oxide is practically insoluble in 70% aq. ethanol or in water, the only possibility for the presence of magnesium in the product is by forming a salt with asiatic acid and madecassic acid.

Further, Centella asiatica extract comprising magnesium salt or complex of asiatic acid and madecassic acid along with asiaticoside and madecassoside showed a pH of about 7.2, whereas Centella asiatica extract comprising asiatic acid and madecassic acid along with asiaticoside and madecassoside showed pH of about 4.5. The difference in pH value clearly suggests the formation of magnesium salt with asiatic acid and madecassic acid.

The inventors surprisingly found that the Centella asiatica extract comprising magnesium salt of asiatic acid and madecassic acid along with asiaticoside and madecassoside (CAMg-1) exhibits better nootropic activities, including cognitive function-related activities compared to the Centella asiatica extract without magnesium (CA-1).

Evaluation of the Centella asiatica Extracts Containing Magnesium and Centella asiatica Extracts without Magnesium for Nootropic Activity (Enhanced Cognitive Function, Reduced Reaction Time, and Increased Alertness) in an In Vivo Model of Sprague Dawley Rats.

In the T-maze reward alternation test, the working memory was significantly improved in the rats supplemented with a blend (CAMg-1) of 70% aq. ethanol and water extracts obtained sequentially from Centella asiatica aerial parts and containing magnesium salt of asiatic acid and madecassic acid along with asiaticoside and madecassoside when compared to the Centella asiatica extracts without magnesium (CA-1). As shown in Table-1, the CAMg-1 supplemented rats (G3 group) showed a 36.1% increase over the control animals. In contrast, the CA-1-supplemented rats (G2 group) showed a 21.3% increase. This observation manifests that the extract blend containing the magnesium is significantly more efficacious than the similar extract blend without the magnesium (Example 6). Similarly, Centella asiatica aerial parts 70% aq. ethanol extract containing magnesium salt of asiatic acid and madecassic acid along with asiaticoside and madecassoside (CAMg-2) also showed better efficacy than the extract without magnesium (CA-2) as summarized in Table 1 below.

The working memory index is calculated using the following formula.

Working memory index=number of correct choices/numbers of total trials or attempts

TABLE 1
Effect of herbal extracts supplementation on working memory index
of Sprague Dawley rats in T-Maze reward alternation test

| | | Working | |
|---|---|---|---|
| | | memory | % increase |
| Animal groups | Dose | index | over control |
| G1 (Control) | 0.5% CMC | 0.61 | — |
| G2 (CA-1) | 50 mg | 0.74 | 21.3 |
| G3 (CAMg-1) | 50 mg | 0.83 | 36.1 |
| G4 (CA-2) | 50 mg | 0.70 | 14.6 |
| G5 (CAMg-2) | 50 mg | 0.78 | 27.9 |

Note:
Higher working memory index values indicate better efficacy

T-maze orientation response test (ORT) is a psychometric test for attention-related behavior in laboratory animals, including rats. A reduced reaction time indicates improved attention and focus in the experimental animals. In this experiment, the reaction time was significantly reduced in the rats supplemented with a blend of Centella asiatica aerial parts extracts (CAMg-1) obtained sequentially with 70% aq. ethanol and water extracts containing magnesium salt of asiatic acid and madecassic acid along with asiaticoside and madecassoside, when compared to the blend of Centella asiatica extracts without magnesium (CA-1). As shown in Table-2, the CAMg-1-supplemented rats (G3 group) showed a 33% reduction in reaction time over the control animals (G1 group); in contrast, the CA-1 supplemented rats (G2 group) showed only a 23% reduction compared to G1 rats. The observation of greater efficacy in the rats supplemented with magnesium containing extract is surprising. Similarly, Centella asiatica aerial parts 70% aq. ethanol extract containing magnesium salt of asiatic acid and madecassic acid along with asiaticoside and madecassoside (CAMg-2) also showed significantly reduced reaction time than the reduction shown by 70% aq. ethanol extract without magnesium (CA-2). Overall, supplementation of magnesium containing extracts (CAMg-1 and CAMg-2) demonstrate greater efficacy in enhancing cognitive function and increasing attention and focus than the corresponding extracts without magnesium (CA-1 and CA-2) in the experimental rats.

TABLE 2
Effect of herbal extracts supplementation on the Reaction time
of Sprague Dawley rats in Orientation Response Test (ORT)

| | | Reaction | % reduction |
|---|---|---|---|
| Animal groups | Dose | time (sec.) | over control |
| G1 (Control) | 0.5% CMC | 3.43 | — |
| G2 (CA-1) | 50 mg | 2.62 | 23 |
| G3 (CAMg-1) | 50 mg | 2.29 | 33 |
| G4 (CA-2) | 50 mg | 2.75 | 19 |
| G5 (CAMg-2) | 50 mg | 2.39 | 30 |

Note:
Lower reaction time values indicate better efficacy

Hence, the inventive Centella asiatica extract comprising magnesium salt of asiatic acid and madecassic acid along with asiaticoside and madecassoside, wherein the total asiaticosides (centellosides) are in the range of 2-40%, and magnesium is in the range of 0.2-5.0%, surprisingly showed better efficacy in nootropic activities compared to the regular extract without magnesium salt.

### Compositions

Encouraged by the increased efficacy in nootropic activities of Centella asiatica extract comprising magnesium salt of asiatic acid and madecassic acid along with asiaticoside and madecassoside, the inventors prepared several compositions of Centella asiatica extract comprising magnesium in combination with at least one ingredient selected from the extracts, fractions, phytochemicals or mixtures thereof derived from Theobroma cacao.

Thus, various solvent extracts of Theobroma cacao seed were prepared with different solvents.

Theobroma cacao seed extracts: T. cacao seeds were pulverized, and the powder was extracted separately with various solvents such as 50% aqueous ethanol, 90% aqueous ethanol, ethanol, water, methanol and acetone to obtain T cacao seed 50% aqueous ethanol extract (Th-1), 90% aqueous ethanol extract (Th-2), ethanol extract (Th-3), water extract (Th-4), methanol extract (Th-5) and acetone extract (Th-6) respectively and these extracts were standardized to theobromine by analytical HPLC method and the details are summarized in examples 7-10.

Then, various compositions (C1-C32) of Centella asiatica extract comprising magnesium salt of asiatic acid and madecassic acid along with asiaticoside and madecassoside; in combination with at least one ingredient selected from the extracts, fractions, phytochemicals or mixtures thereof derived from Theobroma cacao were prepared as described in examples 11-20.

Acetylcholinesterase (AChE): Acetylcholinesterase (AChE) is a cholinergic enzyme that is primarily found at the neuromuscular junctions, especially in muscles and nerves. AChE hydrolyzes Acetylcholine (Ach) into acetic acid and choline. The primary role of AChE is to terminate neuronal transmission and signalling between synapses. Activation of AChE decreases the Ach levels in the brain and is implicated in the pathophysiology of cognitive dysfunction, leading to neurodegenerative disorders such as AD. Hence, inhibition of AChE results in reduced Ach breakdown and it's improved levels in the brain. This increased Ach levels activate the nicotinic and muscarinic receptors that help provide therapeutic relief from cognitive dysfunctions and improve memory, learning, recall information/communication, focus, concentration, reasoning, and problem solving. Several AChE inhibitors such as Tacrine, Donepezil, Rivastigmine, Galantamine have demonstrated clinical efficacy in reducing the symptoms of cognitive dysfunctions and, thus, improve cognitive capabilities and executive functions.

The compositions mentioned above (compositions-1 to 32) were evaluated for acetylcholinesterase (AChE) inhibitory activity in comparison with the corresponding individual ingredients (Example 21). The data from in vitro AChE assay showed that these compositions unexpectedly showed better efficacy in the enzyme inhibition, when compared to their corresponding individual ingredients, suggesting that the compositions containing Centella asiatica extract comprising magnesium salt of asiatic acid and madecassic acid along with asiaticoside and madecassoside in combination with Theobroma cacao extracts tend to exhibit synergism.

For example, the Centella asiatica extract, which is a blend of 70% aq. ethanol and water extract with magnesium (CAMg-1) at 12.5 μg/mL; and Theobroma cacao seed 50% aqueous ethanol extract (Th-1) at 12.5 μg/mL concentration showed 13.75% and 6.70% AChE inhibitions respectively. Surprisingly, the composition-3 containing these two extracts at 1:1 ratio showed 41.20% AChE inhibition at 25 μg/mL concentration, which is significantly better than the additive effect (13.75%+6.70%=20.45%) from these two ingredients, suggesting a synergistic effect between Centella asiatica extract blend of 70% aq. ethanol and water extract with magnesium (CAMg-1) and Theobroma cacao extract (Th-1) in inhibiting AChE. In comparison, a similar composition (Composition-3A) containing Centella asiatica extract blend of 70% aq. ethanol and water extract without magnesium (CA-1) and Theobroma cacao extract (Th-1) in the same ratio of 1:1 showed 29.45% increase at 25 μg/mL, which is much lower than the similar composition (Composition-3) containing the magnesium complex. This manifests a synergistic effect contributed by magnesium to the composition. Similarly, the compositions-1, 2, 4 and 5 obtained when combining Centella asiatica extract, which is a blend of 70% aq. ethanol and water extract with magnesium (CAMg-1) and Theobroma cacao seed 50% aqueous ethanol extract (Th-1) at ratios 3:1, 2:1, 1:2, and 1:3, respectively, also showed synergism in AChE inhibition compared to the inhibitions shown by their corresponding individual ingredients as summarized in Table 4. This increased inhibition of AChE of the inventive compositions is surprising and unexpected.

Similarly, the compositions 6 to 8 containing Centella asiatica extract blend of 70% aq. ethanol and spent water extracts with magnesium (CAMg-1) and Theobroma cacao seed 90% aqueous ethanol extract (Th-2); compositions-9 to 11 containing Centella asiatica extract blend of 70% aq. ethanol and spent water extracts with magnesium (CAMg-1) and Theobroma cacao seed ethanol extract (Th-3); compositions-12 to 14 containing Centella asiatica extract blend of 70% aq. ethanol and spent water extracts with magnesium (CAMg-1) and Theobroma cacao seed water extract (Th-4) also showed synergistic inhibition of acetylcholinesterase when compared to the inhibitions shown by their corresponding individual ingredients as summarized in Table 5.

Similarly, the compositions 16 and 17 containing Centella asiatica extract blend of 70% aq. ethanol and spent water extracts with magnesium (CAMg-1) and Theobroma cacao seed methanol extract (Th-5); compositions 18 to 20 containing Centella asiatica extract blend of 70% aq. ethanol and spent water extracts with magnesium (CAMg-1) and Theobroma cacao seed acetone extract (Th-6); compositions 21 to 23 containing Centella asiatica 70% aq. ethanol extract containing magnesium (CAMg-2) and Theobroma cacao seed 50% aqueous ethanol extract (Th-1); compositions 24 to 26 containing Centella asiatica water extract containing magnesium (CAMg-3) and Theobroma cacao seed ethanol extract (Th-3); compositions-27 to 29 containing Centella asiatica 70% aq. methanol extract containing magnesium (CAMg-4) and Theobroma cacao seed acetone extract (Th-6); compositions 30 to 32 containing Centella asiatica 70% aq. acetone extract containing magnesium (CAMg-5) and Theobroma cacao seed methanol extract (Th-5); also showed synergism in AChE inhibition, when compared to the inhibitions shown by their corresponding individual ingredients as summarized in Tables 6-8.

Formulations: The synergistic herbal compositions comprising a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; may be formulated with at least one component selected from pharmaceutically or nutraceutically or dietically acceptable excipients, carriers and diluents.

The synergistic compositions can be formulated using a pharmaceutically or nutraceutically or dietically acceptable excipients, carriers and diluents; selected from monosaccharides such as glucose, dextrose, fructose, galactose etc.; Disaccharides such as but not limited to sucrose, maltose, lactose, lactulose, trehalose cellobiose, chitobiose etc.; Polycarbohydrates such as starch and modified starch such as sodium starch glycolate, pre-gelatinized starch, soluble starch, ultrasperse A and other modified starches; Dextrins that are produced by hydrolysis of starch or glycogen such as yellow dextrin, white dextrin, maltodextrin, glucidex 12D, rice maltodextrin, tapioca maltodextrin etc.; Polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide, syloids etc.; metallic stearates such as but not limited to calcium stearate, magnesium stearate, zinc stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; Natural pigments, flavors, Class I & Class II preservatives and aqueous, alcoholic, hydro-alcoholic, organic solutions of above listed ingredients alone or in combination.

For example, compositions 33-35 were prepared by mixing a blend of 70% aq. ethanol extract and spent water extract of Centella asiatica aerial parts that were obtained sequentially and containing magnesium salt of asiatic acid and madecassic acid along with asiaticoside and madecassoside (CAMg-1) and Theobroma cacao seed 50% aqueous ethanol extract (Th-1) in three ratios (2:1, 1:1 and 1:2) along with excipients such as rice maltodextrin and colloidal silicon dioxide in a suitable solvent and then evaporating the solvent (example 22).

Process: The process for preparation of the compositions comprising the combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents; which comprises the following steps of;
(a) Extracting dried Centella asiatica powder with suitable solvent;
(b) treating the solution of step (a) with a magnesium source;
(c) filtering the solution of step (b) to obtain the first extract and spent Centella asiatica powder,
(d) extracting the spent Centella asiatica powder with a second solvent;
(e) treating the solution of step (d) with a magnesium source;
(f) filtering the solution of step (e) to obtain the second extract;
(g) combining both extracts obtained in steps c and f;
(h) evaporating the solvent and drying the residue to obtain Centella asiatica extract blend containing magnesium salt or complex of asiatic acid and madecassic acid along with asiaticoside and madecassoside;
(i) optionally, evaporating the filtrate of step-c by omitting steps d to h and drying the residue to obtain Centella asiatica extract containing magnesium salt or complex of asiatic acid and madecassic acid; along with asiaticoside and madecassoside;
(j) blending of extract of step h or step i with Theobroma cacao extract in the presence of a suitable solvent; and optionally with suitable excipients and
(k) drying the product to get the required composition.

The suitable solvents used in the process for the preparation of Centella asiatica extract containing magnesium salt or complex of asiatic acid and madecassic acid along with asiaticoside and madecassoside; according to the present invention is selected from but not limited to C1-C5 alcohols, like ethanol, methanol, n-butanol; water and mixtures thereof; ketones selected from acetone, methylisobutyl ketone, chlorinated solvents selected from methylene dichloride and chloroform; water and mixtures thereof; C1-C7 hydrocarbons such as but not limited to hexane, heptane; esters such as but not limited to ethyl acetate and mixtures thereof. The second solvent is selected from water. The magnesium source used in the process is selected from its oxides, hydroxides, or carbonates; examples include magnesium oxide, magnesium hydroxide, and magnesium carbonate.

Similarly, the suitable solvents used in the process for the preparation of Theobroma cacao extract; according to the present invention is selected from but not limited to C1-C5 alcohols, like ethanol, methanol, n-butanol; water and mixtures thereof; ketones selected from acetone, methylisobutyl ketone, chlorinated solvents selected from methylene dichloride, chloroform; water and mixtures thereof; C1-C7 hydrocarbons such as but not limited to hexane, heptane; esters such as but not limited to ethyl acetate and mixtures thereof.

After identifying the increased efficacy of Centella asiatica extracts containing magnesium; the compositions comprising Centella asiatica extracts containing magnesium with Theobroma cacao seed extracts in different ratios along with excipients (comp 33-35), and their individual ingredients were evaluated for cognition related parameters in an animal model as described below.

Evaluation of the Centella asiatica extracts containing magnesium and Theobroma cacao seed extracts and their compositions for nootropic activity (enhanced cognitive function, reduced reaction time, and increased alertness) in Sprague-Dawley rats: The data from in vivo study expectedly showed better efficacy for the compositions in increasing Nootropic activity when compared to their corresponding individual ingredients further confirming that the Centella asiatica extracts containing magnesium and Theobroma cacao seed extract have the tendency to exhibit synergism when combined (Example 23).

In the T-maze reward alternation test, the working memory was significantly improved in the rats supplemented with compositions containing 1) a blend of sequentially obtained Centella asiatica 70% aq. ethanol extract and spent water extract containing magnesium salt of asiatic acid and madecassic acid along with asiaticoside and madecassoside (CAMg-1) and 2) Theobroma cacao seed 50% aqueous ethanol extract (Th-1), when compared to the individual ingredients.

For example, the Centella asiatica extract containing magnesium (CAMg-1) and Theobroma cacao seed 50% aqueous ethanol extract (Th-1) each at 50 mg dose showed 29.8% and 24.5% increases in working memory index over the control animals, respectively. The composition-33, which contain these two extracts at 2:1 ratio showed 45.8%, which is a significantly higher increase compared to those shown by individual ingredients, thus, suggesting a synergistic effect between these two extracts. Similarly, the compositions 34 and 35 obtained when combining these two ingredients at 1:1 and 2:1, respectively, also showed synergism when compared to their corresponding individual ingredients, as summarized in Table 9.

Further, the compositions (Comp 33-35) comprising Centella asiatica extracts containing magnesium (CAMg-1) with Theobroma cacao seed aqueous 50% ethanol extract (Th-1) have also shown synergistic efficacies in reducing the reaction time in the T-maze orientation response test (ORT), as shown in Table 10.

Together, these data clearly show that compositions 33-35 confer synergistically enhanced cognitive function, reduced reaction time, and increased alertness in the rats.

These compositions also showed synergistic efficacy in increasing three key neurohormones in the brain tissue and serum of the experimental animals compared to the animals supplemented with the individual extracts as explained below.

Norepinephrine (NE), also known as noradrenaline, is an excitatory neurotransmitter produced by the brainstem, hypothalamus, and adrenal glands and released into the bloodstream. Norepinephrine is biosynthesized from dopamine by the action of dopamine β-monooxygenase, predominantly inside the neurotransmitter vesicles. Norepinephrine increases motor-neuron function. An elevated level of norepinephrine in the brain increases alertness and wakefulness.

Dopamine (DA) is a neurotransmitter secreted by the neurons of the substantia nigra located in the midbrain. It is considered a special type of neurotransmitter because of its excitatory and inhibitory effects depending on the type of receptor that dopamine binds to. Dopamine influences many systems of the central nervous system, ranging from movement control to cognitive executive functions and emotional limbic activity. Collectively, dopamine and norepinephrine are involved in major functions such as sensory processing, motor planning, plasticity, and memory encoding in the brain. They are also crucial in mood maintenance, motivation, and concentration or focus. Increased levels of these two key neurotransmitters (neurohormones) are essential for improved sports performance and cognitive functions.

Serotonin (5-hydroxytryptamine, 5-HT) is an inhibitory neurotransmitter involved in emotion and mood. It is secreted by the neurons of the brainstem and by neurons that innervate the gastrointestinal tract (enteric nervous system). It regulates body temperature, perception of pain, emotions, and sleep cycle. Insufficient secretion of serotonin results in a range of emotional disturbances like depression, anger control problems, and obsessive-compulsive disorder; these effects negatively influence attitude and motivation and down-regulate sports performance.

Interestingly, the composition of the present invention showed synergistically improved levels of above neurotransmitters in supplemented animals. For example, the Centella asiatica extract containing magnesium (CAMg-1) and Theobroma cacao seed 50% aqueous ethanol extract (Th-1) supplemented rats showed 13.1% and 18.4% increase in norepinephrine (NE) levels when compared to the control animals. In contrast, the rats supplemented with composition-33 showed a 27.7% increase in Norepinephrine (NE) level, which is a synergistically improved benefit compared to efficacy shown by the individual extracts, e.g., CAMg-1 and Th-1 (Table 11). Similarly, the compositions 34 and 35 exhibited synergistic increases in NE concentration compared to the corresponding individual ingredients.

Further, the compositions (Comp: 33-35) comprising Centella asiatica extracts containing magnesium (CAMg-1) with Theobroma cacao seed extract (Th-1) have also shown synergistic efficacies in increasing serotonin and dopamine levels in the supplemented rats, as presented in Table 11.

In the present study, the compositions 33-35 synergistically increased the levels of dopamine and norepinephrine in the brain tissue and serotonin in the serum of the supplemented rats, compared to the rats supplemented with the corresponding individual extracts. In light of the concepts on physiological activities of the above neurotransmitters, the observations from the present animal study suggest that supplementation of compositions 33-35 provides significant and synergistic improvements in cognitive functions, mood, motivation, alertness, and reaction time. Notably, the observations on the biomarkers provide the physiological basis of the nootropic activities of the above compositions.

Thus, the present herbal compositions comprising the combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents; showed increasing nootropic activities that are essential for improved cognitive functions and boost gaming performance.

Therefore, in an important embodiment, the present invention provides synergistic herbal composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; for increasing nootropic activities that are essential for improving cognitive functions and boosting gaming performance.

In one preferred embodiment, the present invention provides synergistic herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one additional component selected from the group consisting of biological agent(s), and Nootropic agent(s); pharmaceutically acceptable active ingredients, vitamins, minerals; pharmaceutically or nutraceutically or dietically acceptable, excipients, carriers or diluents.

In other preferred embodiment, the present invention provides synergistic herbal compositions comprising the combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; wherein the concentration of Centella asiatica extract in the composition varies in the range of 10%-90% by weight, and Theobroma cacao extract in the composition varies in the range of 90%-10% by weight.

In another embodiment, the present invention discloses synergistic herbal compositions as described above, wherein the composition is standardized to at least one Centella asiatica phytochemical marker selected from asiatic acid, madecassic acid, asiaticoside, and madecassoside or total asiaticosides; and to at least one Theobroma cacao phytochemical reference marker compound or pharmacologically active marker but not limited to theobromine.

In another embodiment, the present invention provides synergistic herbal compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; wherein the total asiaticosides (centellosides) are in the range of 2.0-40%, and magnesium content is in the range of 0.2-5.0%.

In another embodiment, the present invention discloses synergistic herbal compositions as described above, wherein the compositions contain asiatic acid in the range of 0.1-8%, madecassic acid in the range of 0.1-8%, asiaticoside in the range of 0.1-11%, madecassoside in the range of 0.1-13% and magnesium in the range of 0.2-5.0%.

In another embodiment, the present invention discloses synergistic herbal compositions as described above, wherein the composition is standardized to at least one Centella asiatica phytochemical marker selected from asiatic acid, madecassic acid, asiaticoside, and madecassoside or total asiaticosides; and to at least one Theobroma cacao phytochemical reference marker compound or pharmacologically active marker but not limited to theobromine; wherein phytochemical marker compound or group of phytochemical compounds is in the concentration range of 0.01% to 50% by weight of the extract. For example, the Theobroma cacao extract, fraction, or mixtures thereof, is standardized to theobromine and is in the range of 0.5% and 50%.

In another embodiment, the compositions as disclosed above, wherein the phytochemical reference marker compound or pharmacologically active marker or group of markers or its magnesium salt, may be estimated by suitable analytical technique known in the art such as but not limited to HPLC, gravimetry, UV, GC and ICP-mass.

In yet another embodiment, the present invention provides synergistic herbal compositions as described above; wherein the Centella asiatica and Theobroma cacao extracts or their fractions are obtained from at least one plant part selected from the group comprising leaves, stems, tender stems, tender twigs, aerial parts, whole fruit, fruit peel, fruit rind, immature fruit, seeds, flower heads, root, bark, hardwood, rhizome or whole plant or mixtures thereof.

In another embodiment, the present invention provides synergistic herbal compositions as disclosed above, wherein the extracts or fractions or mixtures thereof; are produced using at least one solvent selected from C1-C5 alcohols selected from ethanol, methanol, n-propanol, isopropyl alcohol; ketones selected from acetone, methylisobutyl ketone; chlorinated solvents selected from methylene dichloride and chloroform; water and mixtures thereof; C1-C7 hydrocarbons such as but not limited to hexane, heptane; esters such as but not limited to ethyl acetate and mixtures thereof.

In other embodiment, the process of extraction of Centella asiatica and Theobroma cacao involves the use of techniques known in the art such as maceration, digestion, decoction, infusion, percolation, Soxhlet extraction, refluxing, continuous, ultrasound-assisted, microwave-assisted, pressurized liquid/fluid extraction, supercritical carbon dioxide extraction. Further, the extracts or fractions may be further purified using column chromatography or resin chromatography using a microporous resin.

In another embodiment, the present invention discloses a process for the preparation of compositions comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents,

In yet another embodiment, the present invention provides method of obtaining at least one benefit related to cognitive function, mental ability and/or nootropic activity selected from alertness, vigilance, attention, focus/concentration, neuromotor function (reaction time), memory (verbal, working, visual, logical), intelligence, learning, acquisition, retention, recall information/communication, perception, reasoning, problem solving, decision making, and; reducing mental fatigue and improving gaming performance; wherein the method comprises supplementing the human with an effective dose of a composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected s from pharmaceutically or nutraceutically or dietically acceptable excipients, carriers and diluents.

In yet another embodiment, the present invention provides use of a synergistic herbal composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents; for obtaining at least one benefit related to cognitive function, mental ability and/or nootropic activity selected from alertness, vigilance, attention, focus/concentration, neuromotor function (reaction time), memory (verbal, working, visual, logical), intelligence, learning, acquisition, retention, recall information/communication, perception, reasoning, problem solving, decision making, and; reducing mental fatigue and improving gaming performance.

In another embodiment of the invention, the composition as disclosed above is formulated into a dosage form selected from dry powder form, liquid form, beverage, food product, dietary supplement, or any suitable form such as a tablet, a capsule, a soft chewable tablet, gummies or gummy bar.

In another embodiment of the invention, the composition as disclosed above is formulated into a nutritional/dietary supplement that can be contemplated/made into the dosage form of healthy foods or food for specified health uses such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

In another embodiment of the invention, the composition as disclosed above is formulated into a controlled-release tablet, using controlled-release polymer-based coatings by the techniques including nanotechnology, microencapsulation, hot-melt extrusion, colloidal carrier systems, and other drug delivery systems for obtaining the desired therapeutic benefit.

Those of ordinary skilled in the art will appreciate that changes could be made to the embodiments described above without departing from the broad inventive concept thereof. It is understood, therefore, that this invention is not limited to the particular embodiments or examples disclosed herein but is intended to cover modifications within the objectives and scope of the present invention as defined in the specification. The examples are given solely for illustration and are not to be construed as limitations of the present disclosure, as many variations thereof possible without departing from the spirit of the disclosure.

### EXAMPLES

### Example 1: Preparation of Centella asiatica Aerial Parts Extract, which is a Blend of Sequentially Extracted 70% Aq. Ethanol and Spent Water Extracts Containing Magnesium Salt of Asiatic Acid and Madecassic Acid Along with Asiaticoside and Madecassoside (CAMg-1)

Dried Centella asiatica aerial parts raw material was pulverized, and the powder (50 g) was extracted with 70% aq. ethanol (400 mL) under a hot condition for 2 h. The mixture was filtered, and the extraction process was repeated with 70% aq. ethanol (2×300 mL). To the combined 70% aq. ethanol extract was added magnesium oxide (280 mg) and the mixture was stirred at ambient temperature for 2 h. The solution was filtered, and the filtrate was evaporated under reduced pressure to a minimum volume. The spent raw material was extracted with water (400 mL) at ambient temperature for 3 h. The mixture was filtered, and the extraction process was repeated again with water (2×300 mL). To the combined water extract was added magnesium oxide (90 mg), and the mixture was stirred at ambient temperature for 2 h. The mixture was filtered, and the filtrate was combined with concentrated 70% aq. ethanol extract. The combined extract was evaporated under reduced pressure to obtain Centella asiatica extract containing magnesium (CAMg-1) as brown color solid (15.0 g).

The product was analyzed for asiatic acid, madecassic acid, asiaticoside, and madecassoside (centellosides) by analytical HPLC and magnesium by ICP-MS. It was found to contain 1.36% of asiatic acid, 2.20% of madecassic acid, 3.59% of asiaticoside and 5.88% of madecassoside (i.e., a total of 13.03% of centellosides) and 1.09% of magnesium.

Comparative Example 1A (CA-1): The comparative Centella asiatica extract (CA-1), which is a blend of sequentially extracted 70% aq. ethanol and water extracts containing asiatic acid and madecassic acid along with asiaticoside and madecassoside was prepared as described in example 1, without the step of magnesium oxide treatment.

The product (CA-1) was analyzed for asiatic acid, madecassic acid, asiaticoside and madecassoside (centellosides) by analytical HPLC and magnesium by ICP-MS and it was found to found to contain 1.23% of asiatic acid, 1.96% of madecassic acid, 2.82% of asiaticoside and 4.50% of madecassoside (i.e. a total of 10.51% of centellosides).

### Example 2: Preparation of Centella asiatica Aerial Parts 70% Aq. Ethanol Extract Containing Magnesium Salt of Asiatic Acid and Madecassic Acid Along with Asiaticoside and Madecassoside (CAMg-2)

Dried Centella asiatica aerial parts of raw material were pulverized, and the powder (50 g) was extracted with 70% aq. ethanol (400 mL) at hot condition for 2 h. The mixture was filtered and the extraction process was repeated with 70% aq. ethanol (2×300 mL). The combined 70% aq. ethanol extract was added magnesium oxide (280 mg) and was stirred at ambient temperature for 2 h. The s mixture was filtered, and the filtrate was evaporated under reduced pressure to give the product (CAMg-2) as a brown color solid (11.3 g).

The product was analyzed for asiatic acid, madecassic acid, asiaticoside and madecassoside (centellosides) by analytical HPLC and magnesium by ICP-MS and it was found to contain 1.85% of asiatic acid, 2.13% of madecassic acid, 3.76% of asiaticoside and 5.95% of madecassoside (i.e. a total of 13.69% of centellosides) and 0.61% of magnesium.

Comparative Example 2A (CA-2): The comparative Centella asiatica aerial parts 70% aq. ethanol extract (CA-2) was prepared as described in example 2, without the step of magnesium oxide treatment.

The product was analyzed for asiatic acid, madecassic acid, asiaticoside and madecassoside (centellosides) by analytical HPLC and magnesium by ICP-MS and found to contain 1.46% of asiatic acid, 2.34% of madecassic acid, 3.56% of asiaticoside and 5.53% of madecassoside (i.e. a total of 12.88% of centellosides).

### Example 3: Preparation of Centella asiatica Aerial Parts Water Extract Containing Magnesium Salt of Asiatic Acid and Madecassic Acid Along with Asiaticoside and Madecassoside (CAMg-3)

Dried Centella asiatica aerial parts of raw material were pulverized, and the powder (50 g) was extracted with water (400 mL) in hot condition for 2 h. The mixture was filtered, and the extraction process was repeated with water (2×300 mL). To the combined water extract was added magnesium oxide (230 mg), and the mixture was stirred at ambient temperature for 2 h. The mixture was filtered, and the filtrate was evaporated under reduced pressure to give the product (CAMg-3) as a brown color solid (13.0 g).

The product was analyzed for centellosides by HPLC and magnesium by ICP-MS and the extract was found to contain 7.95% of centellosides and 1.45% of magnesium.

### Example 4: Preparation of Centella asiatica Aerial Parts 70% Aq. Methanol Extract Containing Magnesium Salt of Asiatic Acid and Madecassic Acid Along with Asiaticoside and Madecassoside (CAMg-4)

Dried Centella asiatica aerial parts of raw material were pulverized, and the powder (50 g) was extracted with 70% aq. methanol (400 mL) in hot condition for 2 h. The mixture was filtered and the extraction process was repeated with 70% aq. methanol (2×300 mL). To the combined 70% aq. methanol extract was added magnesium oxide (280 mg) and the mixture was stirred at ambient temperature for 2 h. The mixture was filtered, and the filtrate was evaporated under reduced pressure to give the product (CAMg-4) as a brown color solid (14.2 g).

The product was analyzed for asiatic acid, madecassic acid, asiaticoside and madecassoside (centellosides) by analytical HPLC and magnesium by ICP-MS and it was found to contain 2.86% of asiatic acid, 2.01% of madecassic acid, 0.99% of asiaticoside and 0.58% of madecassoside (i.e. a total of 6.44% of centellosides) and 1.02% of magnesium.

### Example 5: Preparation of Centella asiatica Aerial Parts 70% Aq. Acetone Extract Containing Magnesium Salt of Asiatic Acid and Madecassic Acid Along with Asiaticoside and Madecassoside (CAMg-5)

Dried Centella asiatica aerial parts of raw material were pulverized, and the powder (50 g) was extracted with 70% aq. acetone (400 mL) in hot condition for 2 h. The mixture was filtered and the extraction process was repeated with 70% aq. acetone (2×300 mL). To the combined 70% aq. acetone extract was added magnesium oxide (280 mg) and the mixture was stirred at ambient temperature for 2 h. The mixture was filtered, and the filtrate was evaporated under reduced pressure to give the product (CAMg-5) as a brown color solid (5.8 g).

The product was analyzed for asiatic acid, madecassic acid, asiaticoside and madecassoside (centellosides) by analytical HPLC and magnesium by ICP-MS and found to contain 3.23% of asiatic acid, 3.13% of madecassic acid, 4.33% of asiaticoside and 2.90% of madecassoside (i.e. a total of 13.58% of centellosides) and 1.21% of magnesium.

### Example 6: Evaluation of the Herbal Extracts for Nootropic Activity in Sprague-Dawley Rats

Treatments: Thirty healthy male Sprague-Dawley rats (Age: 6-8 weeks) were randomly assigned to five groups (n=6). The animals were supplemented (using oral gavage) daily with 50 mg/kg of CA-1 (G2) or CAMg-1 (G3) or CA-2 (G4) or CAMg-2 (G5) in 10 mL of 0.5% CMC in water, for 30 days. The rats in the control (G1) group received only the vehicle (10 mL of 0.5% CMC in water) during the study. T-maze test was conducted on all animals on day 31 of the study.

Test parameters: T-maze reward alternation test—working memory and orientation response test (ORT)—attention & reaction time were evaluated as per the procedures disclosed in the literature, and the results are presented in Tables-1 & 2. The working memory index is calculated using the following formula.

Working memory index=number of correct choices/number of total trials or attempts

TABLE 1
Effect of herbal extracts supplementation on working memory index
of Sprague Dawley rats in T-Maze reward alternation test

| | | Working | |
|---|---|---|---|
| | | memory | % increase |
| Animal groups | Dose | index | over control |
| G1 (Control) | 0.5% CMC | 0.61 | — |
| G2 (CA-1) | 50 mg | 0.74 | 21.3 |
| G3 (CAMg-1) | 50 mg | 0.83 | 36.1 |
| G4 (CA-2) | 50 mg | 0.70 | 14.6 |
| G5 (CAMg-2) | 50 mg | 0.78 | 27.9 |

Note:
Higher working memory index values indicate better efficacy

TABLE 2
Effect of herbal extracts supplementation on the Reaction time
of Sprague Dawley rats in Orientation Response Test (ORT)

| | | Reaction | % reduction |
|---|---|---|---|
| Animal groups | Dose | time (sec.) | over control |
| G1 (Control) | 0.5% CMC | 3.43 | — |
| G2 (CA-1) | 50 mg | 2.62 | 23 |
| G3 (CAMg-1) | 50 mg | 2.29 | 33 |
| G4 (CA-2) | 50 mg | 2.75 | 19 |
| G5 (CAMg-2) | 50 mg | 2.39 | 30 |

Note:
Lower reaction time values indicate better efficacy

### Example 7: Preparation of Theobroma cacao Seed 50% Aqueous Ethanol Extract (Th-1)

Theobroma cacao seeds (100 g) were pulverized, and the powder was extracted with 50% aq. ethanol (700 mL) in hot condition for 1 h. The extract was filtered, and the spent raw material was re-extracted twice with 50% aq. ethanol (2×500 mL) under similar conditions. The combined extract was filtered and concentrated under vacuum to obtain a residue of 50% aq. ethanol extract (Th-1; 11.2 g).

### Example 8: Preparation of Theobroma cacao Seed 90% Aqueous Ethanol, Ethanol and Water Extracts

The 90% aqueous ethanol extract (Th-2; 4.5 g), ethanol extract (Th-3; 4.1 g) and water extract (Th-4; 12.2 g) of Theobroma cacao seeds were obtained by adopting similar extraction procedure as described in example 7 using 90% aqueous ethanol, ethanol, and water as extraction solvents respectively.

### Example 9: Preparation of Theobroma cacao Seed Methanol and Acetone Extracts

The methanol extract (Th-5; 5.8 g) and acetone extract (Th-6; 27 g) of Theobroma cacao seeds were obtained by adopting similar extraction procedure as described in example 7 using methanol and acetone as extraction solvents respectively.

### Example 10: Standardization of Theobroma cacao Seed Extracts

The various extracts of Theobroma cacao seed were standardized to Theobromine by analytical HPLC method and the results are summarized in Table 3.

TABLE 3
HPLC results of Theobroma cacao seed extracts

| | | Solvent for | Theobromine |
|---|---|---|---|
| S. No. | Extract code | extraction | (HPLC) |
| 1 | Th-1 | 50% ethanol | 6.2% |
| 2 | Th-2 | 90% ethanol | 8.9% |
| 3 | Th-3 | Ethanol | 7.1% |
| 4 | Th-4 | water | 3.1% |
| 5 | Th-5 | Methanol | 6.5% |
| 6 | Th-6 | Acetone | 0.79% |

### Example 11: Preparation of Various Compositions Containing Centella asiatica Aerial Parts Extract Blend of Sequentially Obtained 70% Aq. Ethanol and Water Extracts Containing Magnesium Salt of Asiatic Acid and Madecassic Acid Along with Asiaticoside and Madecassoside (CAMg-1) and Theobroma cacao Seed 50% Aqueous Ethanol Extract (Th-1)

Comp-1: C-1 was prepared by combining CAMg-1 and Th-1 in the ratio of 3:1.

Comp-2: C-2 was prepared by combining CAMg-1 and Th-1 in the ratio of 2:1.

Comp-3: C-3 was prepared by combining CAMg-1 and Th-1 in the ratio of 1:1.

Comp-4: C-4 was prepared by combining CAMg-1 and Th-1 in the ratio of 1:2.

Comp-5: C-5 was prepared by combining CAMg-1 and Th-1 in the ratio of 1:3.

Composition for comparison (comp-3A): Comp-3A was prepared by combining Centella asiatica aerial parts extract blend of sequentially obtained 70% aq. ethanol and water extracts without magnesium (CA-1) and Theobroma cacao seed 50% aqueous ethanol extract (Th-1) in the ratio of 1:1.

### Example 12: Preparation of Various Compositions Containing Centella asiatica Aerial Parts Extract Blend of 70% Aq. Ethanol and Water Extract Containing Magnesium Salt of Asiatic Acid and Madecassic Acid Along with Asiaticoside and Madecassoside Obtained Sequentially (CAMg-1) and Theobroma cacao Seed 90% Aqueous Ethanol Extract (Th-2)

Comp-6: C-6 was prepared by combining CAMg-1 and Th-2 in the ratio of 2:1.

Comp-7: C-7 was prepared by combining CAMg-1 and Th-2 in the ratio of 1:1.

Comp-8: C-8 was prepared by combining CAMg-1 and Th-2 in the ratio of 1:2.

### Example 13: Preparation of Various Compositions Containing Centella asiatica Aerial Parts Extract Blend of 70% Aq. Ethanol and Water Extract Containing Magnesium Salt of Asiatic Acid and Madecassic Acid Along with Asiaticoside and Madecassoside Obtained Sequentially (CAMg-1) and Theobroma cacao Seed Ethanol Extract (Th-3)

Comp-9: C-9 was prepared by combining CAMg-1 and Th-3 in the ratio of 2:1.

Comp-10: C-10 was prepared by combining CAMg-1 and Th-3 in the ratio of 1:1.

Comp-11: C-11 was prepared by combining CAMg-1 and Th-3 in the ratio of 1:2.

### Example 14: Preparation of Various Compositions Containing Centella asiatica Aerial Parts Extract Blend of 70% Aq. Ethanol and Water Extract Containing Magnesium Salt of Asiatic Acid and Madecassic Acid Along with Asiaticoside and Madecassoside Obtained Sequentially (CAMg-1) and Theobroma cacao Seed Water Extract (Th-4)

Comp-12: C-12 was prepared by combining CAMg-1 and Th-4 in the ratio of 2:1.

Comp-13: C-13 was prepared by combining CAMg-1 and Th-4 in the ratio of 1:1.

Comp-14: C-14 was prepared by combining CAMg-1 and Th-4 in the ratio of 1:2.

### Example 15: Preparation of Various Compositions Containing Centella asiatica Aerial Parts Extract Blend of 70% Aq. Ethanol and Water Extract Containing Magnesium Salt of Asiatic Acid and Madecassic Acid Along with Asiaticoside and Madecassoside Obtained Sequentially (CAMg-1) and Theobroma cacao Seed Methanol Extract (Th-5)

Comp-15: C-15 was prepared by combining CAMg-1 and Th-5 in the ratio of 2:1.

Comp-16: C-16 was prepared by combining CAMg-1 and Th-5 in the ratio of 1:1.

Comp-17: C-17 was prepared by combining CAMg-1 and Th-5 in the ratio of 1:2.

### Example 16: Preparation of Various Compositions Containing Centella asiatica Aerial Parts Extract Blend of 70% Aq. Ethanol and Water Extract Containing Magnesium Salt of Asiatic Acid and Madecassic Acid Along with Asiaticoside and Madecassoside Obtained Sequentially (CAMg-1) and Theobroma cacao Seed Acetone Extract (Th-6)

Comp-18: C-18 was prepared by combining CAMg-1 and Th-6 in the ratio of 2:1.

Comp-19: C-19 was prepared by combining CAMg-1 and Th-6 in the ratio of 1:1.

Comp-20: C-20 was prepared by combining CAMg-1 and Th-6 in the ratio of 1:2.

### Example 17: Preparation of Various Compositions Containing Centella asiatica Aerial Parts 70% Aq. Ethanol Extract Containing Magnesium Salt of Asiatic Acid and Madecassic Acid Along with Asiaticoside and Madecassoside (CAMg-2) and Theobroma cacao Seed 50% Aqueous Ethanol Extract (Th-1)

Comp-21: C-21 was prepared by combining CAMg-2 and Th-1 in the ratio of 2:1.

Comp-22: C-22 was prepared by combining CAMg-2 and Th-1 in the ratio of 1:1.

Comp-23: C-23 was prepared by combining CAMg-2 and Th-1 in the ratio of 1:2.

### Example 18: Preparation of Various Compositions Containing Centella asiatica Aerial Parts Water Extract Containing Magnesium Salt of Asiatic Acid and Madecassic Acid Along with Asiaticoside and Madecassoside (CAMg-3) and Theobroma cacao Seed Ethanol Extract (Th-3)

Comp-24: C-24 was prepared by combining CAMg-3 and Th-3 in the ratio of 2:1.

Comp-25: C-25 was prepared by combining CAMg-3 and Th-3 in the ratio of 1:1.

Comp-26: C-26 was prepared by combining CAMg-3 and Th-3 in the ratio of 1:2.

### Example 19: Preparation of Various Compositions Containing Centella asiatica Aerial Parts 70% Aq. Methanol Extract Containing Magnesium Salt of Asiatic Acid and Madecassic Acid Along with Asiaticoside and Madecassoside (CAMg-4) and Theobroma cacao Seed Acetone Extract (Th-6)

Comp-27: C-27 was prepared by combining CAMg-4 and Th-6 in the ratio of 2:1.

Comp-28: C-28 was prepared by combining CAMg-4 and Th-6 in the ratio of 1:1.

Comp-29: C-29 was prepared by combining CAMg-4 and Th-6 in the ratio of 1:2.

### Example 20: Preparation of Various Compositions Containing Centella asiatica Aerial Parts 70% Aq. Acetone Extract Containing Magnesium Salt of Asiatic Acid and Madecassic Acid Along with Asiaticoside and Madecassoside (CAMg-5) and Theobroma cacao Seed Methanol Extract (Th-5)

Comp-30: C-30 was prepared by combining CAMg-5 and Th-5 in the ratio of 2:1.

Comp-31: C-31 was prepared by combining CAMg-5 and Th-5 in the ratio of 1:1.

Comp-32: C-32 was prepared by combining CAMg-5 and Th-5 in the ratio of 1:2.

### Example 21: General Procedure for Acetylcholinesterase Inhibitory Activity

Acetylcholinesterase activity was measured using the substrate acetylthiocholine iodide, which converts to thiocholine. The reaction of thiocholine with the chromogenic substrate Dithionitrobenzoic acid (DTNB) leads to the formation of a yellow anion of 2-thio-2-Nitrobenzoic acid, which absorbs strongly at 412 nm.

The AChE assay was performed by the method of Ellman et. al, with minor modifications, using acetylthiocholine iodide as a substrate (Lee J. H., et. al. Arch Pharm Res 2004, 27(1): 53-56). Ellmans reaction mixture contains, 1.0 mM acetylthiocholine iodide and 0.5 mM 5, 5′-dithio-bis-(2-nitrobenzoic acid) in a 50 mM sodium phosphate buffer (pH 8.0). The assay mixture contained 50 μl of 50 mM phosphate buffer at pH—8.0, 30 μl of test substance at various concentrations and 20 μl of (100 mU/mL) enzyme. For blanks, the enzyme was replaced with phosphate buffer. The reaction mixture was mixed thoroughly, 100 μl of Ellman's reagent was added and incubated at room temperature for 10 min. The absorbance was measured at 412 nm using microplate reader. The percentage inhibition of enzyme activity was calculated by comparing OD's of tests wells with that of control wells using the following formula. Calculations: % inhibition=[(control−sample)/control]×100. The results of Acetylcholinesterase inhibitory activity of the individual extracts and their compositions are summarized in Tables 4-8.

TABLE 4
Acetylcholinesterase inhibitory activities of the compositions containing
blend of Centella asiatica aerial parts extracts, derived sequentially
using 70% aq. ethanol and water, with magnesium (CAMg-1) or without magnesium
(CA-1) and Theobroma cacao seed 50% aqueous ethanol extract (Th-1)

| | | | | | | | % Inhibition of AChE | |
|---|---|---|---|---|---|---|---|---|
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | CAMg-1 | | Th-1 | | | | | |
| C-1 | 18.75 | 20.63 | 6.25 | 9.38 | 3:1 | 25 | 30.00 | 37.43 |
| C-2 | 16.67 | 18.34 | 8.33 | 9.47 | 2:1 | 25 | 27.81 | 39.39 |
| C-3 | 12.5 | 13.75 | 12.5 | 6.70 | 1:1 | 25 | 20.45 | 41.20 |
| C-4 | 8.33 | 9.16 | 16.67 | 12.80 | 1:2 | 25 | 21.97 | 35.75 |
| C-5 | 6.25 | 6.88 | 18.75 | 9.56 | 1:3 | 25 | 16.44 | 31.17 |
| | CA-1 | | Th-1 | | | | | |
| C-3A | 12.5 | 12.41 | 12.5 | 18.75 | 1:1 | 25 | 31.16 | 29.45 |

TABLE 5
Acetylcholinesterase inhibitory activities of the compositions containing blend
of Centella asiatica aerial parts extracts, derived sequentially using 70% aq.
ethanol and water, with magnesium (CAMg-1) and Theobroma cacao seed 90% aqueous
ethanol extract (Th-2) or ethanol extract (Th-3) or water extract (Th-4)

| | | | | | | | % Inhibition of AChE | |
|---|---|---|---|---|---|---|---|---|
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | CAMg-1 | | Th-2 | | | | | |
| C-6 | 8.33 | 12.87 | 4.17 | 8.22 | 2:1 | 12.5 | 21.09 | 34.04 |
| C-7 | 6.25 | 9.66 | 6.25 | 12.32 | 1:1 | 12.5 | 21.98 | 32.05 |
| C-8 | 4.17 | 6.44 | 8.33 | 16.42 | 1:2 | 12.5 | 22.86 | 36.36 |
| | CAMg-1 | | Th-3 | | | | | |
| C-9 | 8.33 | 12.87 | 4.17 | 4.96 | 2:1 | 12.5 | 17.84 | 29.72 |
| C-10 | 6.25 | 9.66 | 6.25 | 7.44 | 1:1 | 12.5 | 17.10 | 27.61 |
| C-11 | 4.17 | 6.44 | 8.33 | 9.91 | 1:2 | 12.5 | 16.36 | 24.18 |
| | CAMg-1 | | Th-4 | | | | | |
| C-12 | 8.33 | 12.87 | 4.17 | 2.74 | 2:1 | 12.5 | 15.62 | 24.95 |
| C-13 | 6.25 | 9.66 | 6.25 | 4.11 | 1:1 | 12.5 | 13.77 | 26.46 |
| C-14 | 4.17 | 6.44 | 8.33 | 5.48 | 1:2 | 12.5 | 11.92 | 21.69 |

TABLE 6
Acetylcholinesterase inhibitory activities of the compositions containing
blend of Centella asiatica aerial parts extracts, derived sequentially
using 70% aq. ethanol and water, with magnesium (CAMg-1) and Theobroma cacao
seed methanol extract (Th-5) or acetone extract (Th-6)

| | | | | | | | % Inhibition of AChE | |
|---|---|---|---|---|---|---|---|---|
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | CAMg-1 | | Th-5 | | | | | |
| C-16 | 12.5 | 13.75 | 12.5 | 4.06 | 1:1 | 25 | 17.81 | 25.64 |
| C-17 | 8.33 | 9.16 | 16.67 | 5.41 | 1:2 | 25 | 14.58 | 24.17 |
| | CAMg-1 | | Th-6 | | | | | |
| C-18 | 16.67 | 18.34 | 8.33 | 3.53 | 2:1 | 25 | 21.87 | 32.62 |
| C-19 | 12.5 | 13.75 | 12.5 | 5.30 | 1:1 | 25 | 19.05 | 31.27 |
| C-20 | 8.33 | 9.16 | 16.67 | 7.06 | 1:2 | 25 | 16.22 | 26.15 |

TABLE 7
Acetylcholinesterase inhibitory activities of the compositions containing
Centella asiatica 70% aq. ethanol extract containing magnesium (CAMg-
2) and Theobroma cacao seed 50% aqueous ethanol extract (Th-1); compositions
containing Centella asiatica water extract containing magnesium (CAMg-3)
and Theobroma cacao seed ethanol extract (Th-3)

| | | | | | | | % Inhibition of AChE | |
|---|---|---|---|---|---|---|---|---|
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | CAMg-2 | | Th-1 | | | | | |
| C-21 | 16.67 | 9.42 | 8.33 | 12.50 | 2:1 | 25 | 21.91 | 30.36 |
| C-22 | 12.5 | 7.06 | 12.5 | 18.75 | 1:1 | 25 | 25.81 | 36.70 |
| C-23 | 8.33 | 4.70 | 16.67 | 25.01 | 1:2 | 25 | 29.71 | 40.14 |
| | CAMg-3 | | Th-3 | | | | | |
| C-24 | 16.67 | 21.47 | 8.33 | 7.23 | 2:1 | 25 | 28.70 | 39.82 |
| C-25 | 12.5 | 16.10 | 12.5 | 10.86 | 1:1 | 25 | 26.96 | 37.30 |
| C-26 | 8.33 | 10.73 | 16.67 | 14.48 | 1:2 | 25 | 25.21 | 35.47 |

TABLE 8
Acetylcholinesterase inhibitory activities of the compositions containing Centella asiatica
70% aq. methanol extract containing magnesium (CAMg-4) and Theobroma cacao seed acetone
extract (Th-6); compositions containing Centella asiatica 70% aq. acetone extract containing
magnesium (CAMg-5) and Theobroma cacao seed methanol extract (Th-5)

| | | | | | | | % Inhibition of AChE | |
|---|---|---|---|---|---|---|---|---|
| | | | | | | Comp | production | |
| Comp | | % | | % | | Dose | Additive | |
| # | μg/mL | Inhibition | μg/mL | Inhibition | Ratio | μg/mL | (Calculated) | Observed |
| | CAMg-4 | | Th-6 | | | | | |
| C-27 | 16.67 | 1.48 | 8.33 | 3.53 | 2:1 | 25 | 5.01 | 15.05 |
| C-28 | 12.5 | 1.11 | 12.5 | 5.30 | 1:1 | 25 | 6.41 | 17.59 |
| C-29 | 8.33 | 0.74 | 16.67 | 7.06 | 1:2 | 25 | 7.80 | 15.53 |
| | CAMg-5 | | Th-5 | | | | | |
| C-30 | 16.67 | 17.38 | 8.33 | 2.71 | 2:1 | 25 | 20.08 | 32.17 |
| C-31 | 12.5 | 13.03 | 12.5 | 4.06 | 1:1 | 25 | 17.09 | 36.38 |
| C-32 | 8.33 | 8.68 | 16.67 | 5.41 | 1:2 | 25 | 14.10 | 29.42 |

### Example 22: Formulation of the Compositions

Comp-33 (CAMg-1 and Th-1 in the ratio of 2:1): A mixture of Centella asiatica aerial parts extract blend (CAMg-1, 6.0 Kg) and water (14 L) was stirred at ambient temperature for 10-15 min. Added Theobroma cacao seed 50% aqueous ethanol extract (Th-1, 3.0 Kg) to the mixture slowly and stirred for a further 10-15 min. After mixing the contents thoroughly, rice maltodextrin (0.8 Kg) was added and stirred further for 20-30 min. The contents were dried at ambient temperature under reduced pressure to give the product as flakes. These flakes were homogenously blended with colloidal silicon dioxide (0.2 Kg) in a polyethylene cover or in suitable blender. The material was pulverized further and sifted through the #40 sieve to get the composition-33 as a fine powder.

Comp-34 (CAMg-1 and Th-1 in the ratio of 1:1): A mixture of CAMg-1 (4.5 Kg) and water (10 L) was stirred at ambient temperature for 10-15 min. To the mixture was slowly added Th-1 (4.5 Kg) and stirred for a further 10-15 min. After mixing the contents thoroughly, rice maltodextrin (0.8 Kg) was added and continued the stirring for another 20-30 min. The contents were dried at ambient temperature under reduced pressure to give the product as flakes. These flakes were homogenously blended with colloidal silicon dioxide (0.2 Kg) in a polyethylene cover or in a suitable blender. The material was pulverized further and sifted through the #40 sieve to get the composition-34 as a fine powder.

Comp-35 (CAMg-1 and Th-1 in the ratio of 1:2): A mixture of CAMg-1 (3.0 Kg) and water (10 L) was stirred at ambient temperature for 10-15 min. To the mixture was slowly added Th-1 (6.0 Kg) and stirred for a further 10-15 min. After mixing the contents thoroughly, rice maltodextrin (0.8 Kg) was added and continued the stirring for another 20-30 min. The contents were dried at ambient temperature under reduced pressure to give the product as flakes. These flakes were homogenously blended with colloidal silicon dioxide (0.2 Kg) in a polyethylene cover or in a suitable blender. The material was pulverized further and sifted through the #40 sieve to get the composition-35 as a fine powder.

### Example 23: Evaluation of the Herbal Extracts and their Compositions for Nootropic Activity in Sprague-Dawley Rats

Treatments: Thirty-six healthy male Sprague-Dawley rats (Age: 6-8 weeks) were randomly assigned to six groups (n=6). The animals were supplemented (using oral gavage) with 50 mg/kg of CAMg-1 (G2) or Th-1 (G3) or Comp-33 (G4) or Comp-34 (G5) or Comp-35 (G6) in 10 mL of 0.5% CMC in water, for 30 days. The control (G1) rats received only the vehicle (10 mL of 0.5% CMC in water) during the study. A T-maze test was conducted on all animals on day 31 of the study.

Test parameters: T-maze reward alternation test—working memory and orientation response test (ORT)—attention & reaction time were evaluated as per the procedures disclosed in the literature, and the results are presented in Tables 9 & 10. The working memory index is calculated using the following formula.

Working memory index=number of correct choices/number of total trials or attempts

TABLE 9
Effect of supplementation of herbal extracts and
their compositions on working memory index of Sprague
Dawley rats in T-Maze reward alternation test

| | | Average | |
|---|---|---|---|
| | | Working | % increase |
| Animal groups | Dose | memory index | over control |
| G1 (Control) | 0.5% CMC | 0.57 | — |
| G2 (CAMg-1) | 50 mg | 0.74 | 29.8 |
| G3 (Th-1) | 50 mg | 0.71 | 24.5 |
| G4 (Comp-33, 2:1) | 50 mg | 0.83 | 45.8 |
| G5 (Comp-34, 1:1) | 50 mg | 0.87 | 52.3 |
| G6 (Comp-35, 1:2) | 50 mg | 0.82 | 43.9 |

Note:
Higher the values represent better efficacy

TABLE 10
Effect of supplementation of herbal extracts and
their compositions on the Reaction time of Sprague
Dawley rats in Orientation Response Test (ORT)

| | | Average | |
|---|---|---|---|
| | | Reaction time* | % reduction |
| Animal groups | Dose | (sec.) | over control |
| G1 (Control) | 0.5% CMC | 3.29 | — |
| G2 (CAMg-1) | 50 mg | 2.35 | 28.5 |
| G3 (Th-1) | 50 mg | 2.47 | 24.8 |
| G4 (Comp-33, 2:1) | 50 mg | 1.98 | 39.9 |
| G5 (Comp-34, 1:1) | 50 mg | 1.84 | 44.0 |
| G6 (Comp-35, 1:2) | 50 mg | 2.01 | 38.8 |

*Note:
Lower the values represent better efficacy

Norepinephrine Measurement: Norepinephrine levels were measured in the brain tissue from cortex region. Approximately 30 mg of brain cortex tissue was weighed and collected in 1× phosphate-buffered saline (1×PBS, 270 μL, pH 7.4). The tissue was homogenized using a Dounce homogenizer under ice-cold conditions. After homogenization, the tissue was sonicated for 30 seconds in a probe sonicator. The tissue homogenate was centrifuged at 21952×g for 15 minutes at 4° C. The supernatant was collected, and protein was estimated using a Pierce™ BCA protein assay kit (Cat #23225, Thermo-Fisher Scientific, Waltham, MA). Norepinephrine assay was performed using Elabscience NA/NE (Noradrenaline/Norepinephrine) ELISA kit (Cat #E-EL-0047; Wuhan, China) according to the manufacturer's protocol. Briefly, in 96-well microtiter strips, each 50 μL of standard, blank, and tissue lysate samples were added into appropriate wells. Immediately, 50 μL of biotinylated detection antibody working solution was added to all the wells, plate sealed, and incubated for 45 minutes at 37° C. The microtiter wells were washed 3 times with wash buffer; 100 μL of HRP conjugate solution was added, the plate was sealed, and incubated for 30 minutes at 37° C. After washing (3 times) with wash buffer, 90 μL of substrate reagent was added, the plate was sealed, and incubated for 15 minutes at 37° C. Finally, 50 μL of stop solution was added to each microtiter well, mixed, and absorbance was measured at 450 nm with a correction wavelength of 650 nm in a microplate reader (SpectraMax 5e, Molecular Devices, San Jose, CA). Norepinephrine concentrations in the tissue lysates were determined based on the standard curve plotted using the Norepinephrine standard and normalized with the protein content in the tissue lysates.

Serotonin Measurement: Serotonin levels were measured in the hippocampus region of the brain tissue. Approximately, 30 mg of brain hippocampus tissue was weighed and collected in 1× phosphate buffered saline (1×PBS, 270 μL, pH 7.4). The tissue was homogenized using a Dounce homogenizer under ice-cold conditions. After homogenization, the tissue was sonicated for 30 seconds in a probe sonicator. The tissue homogenate was centrifuged at 21952×g for 15 minutes at 4° C. The supernatant was collected, and protein was estimated using a Pierce™ BCA protein assay kit (Cat #23225, Thermo-Fisher Scientific, Waltham, MA). Serotonin assay was performed using Elabscience ST/5-HT (Serotonin/5-Hydroxytryptamine) ELISA kit (Cat #E-EL-0033; Wuhan, China) according to the manufacturer's protocol. Briefly, in a 96-well microtiter strips, each 50 μL of standards, blank, tissue lysate samples were added into appropriate wells. Immediately, 50 μL of biotinylated detection antibody working solution was added to all the wells, plate sealed, and incubated for 45 minutes at 37° C. The microtiter wells were washed 3 times with wash buffer; 100 μL of HRP conjugate solution was added, the plate was sealed, and incubated for 30 minutes at 37° C. After washing (3 times) with wash buffer, 90 μL of substrate reagent was added, the plate was sealed, and incubated for 15 minutes at 37° C. Finally, 50 μL of stop solution was added to each microtiter well, mixed, and absorbance was measured at 450 nm with a correction wavelength of 650 nm in a microplate reader (SpectraMax 5e Molecular Devices, San Jose, CA). Serotonin concentrations in the tissue lysates were determined based on the standard curve plotted using the Serotonin standard and normalized with the protein content in the tissue lysates.

Dopamine Measurement: Dopamine levels were measured in the serum samples using LDN Dopamine Fast Track ELISA Kit (Cat #BA E-6300; Nordhorn, Germany) according to the manufacturer's protocol. Briefly, Dopamine from the serum samples or standards was extracted and acylated using extraction and acylation buffers provided in the kit. In 96-well Dopamine microtiter strips, each 25 μL of enzyme solution, extracted standards, controls, and extracted serum samples were added and incubated for 30 minutes at room temperature (RT) on a shaker. After the incubation, 25 μL of Dopamine antiserum was added, covered the plate with an adhesive foil, and incubated for 2 hours at RT on a shaker. The microtiter wells were washed 3 times with wash buffer; 100 μL of enzyme conjugate was added and incubated for 30 minutes at RT on a shaker. After washing (3 times) with wash buffer, 100 μL of the substrate was added to the microtiter wells and incubated at ambient temperature (20-25° C.) for 30 minutes on a shaker. Finally, 100 μL of stop solution was added to each microtiter wells, mixed, and absorbance was measured at 450 nm with a correction wavelength of 650 nm in a microplate reader (SpectraMax 5e, Molecular Devices, San Jose, CA). Dopamine concentrations in the serum samples were calculated based on the standard curve plotted using the Dopamine standard. The results are presented in Table 11.

TABLE 11
Bio-markers data

| | Norepinephrine | | Serotonin (ng/mg | | Serum Dopamine | |
|---|---|---|---|---|---|---|
| | (ng/mg protein) | | protein) | | (ng/ml) | |
| | | % of | | % of | | % of |
| | | increase | | increase | | increase |
| | Mean | over | Mean | over | Mean | over |
| Animal groups | Concn. | control | Concn. | control | Concn. | control |
| G1 (Control) | 0.135 | — | 0.108 | — | 11.814 | — |
| G2 (CAMg-1) | 0.153 | 13.1 | 0.128 | 18.5 | 14.212 | 20.3 |
| G3 (Th-1) | 0.160 | 18.4 | 0.123 | 14.1 | 15.263 | 29.2 |
| G4 (Comp-33, 2:1) | 0.172 | 27.7 | 0.132 | 22.1 | 16.492 | 39.6 |
| G5 (Comp-34, 1:1) | 0.183 | 35.4 | 0.136 | 25.7 | 17.237 | 45.9 |
| G6 (Comp-35, 1:2) | 0.178 | 31.9 | 0.131 | 21.3 | 16.622 | 40.7 |

Note:
Higher mean concentration values indicate better efficacy

Although the invention has been described in detail in the foregoing for the purpose of illustration, it is to be understood that such detail is solely for that purpose and that variations can be made therein by those skilled in the art without departing from the spirit and scope of the invention except as it may be limited by the claims

1. A synergistic herbal composition comprising: a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; for increasing nootropic activities that are essential for improving cognitive functions and boosting gaming performance.

2. The synergistic herbal composition as claimed in claim 1, wherein the concentration of Centella asiatica extract varies in the range of 10%-90% by weight, and Theobroma cacao extract varies in the range of 90%-10% by weight.

3. The synergistic herbal composition as claimed in claim 1, wherein the total asiaticosides content is in the range of 1.0-40%.

4. The synergistic herbal composition as claimed in claim 3, wherein the total asiaticosides content comprises of asiatic acid in the range of 0.1%-8.0%, madecassic acid in the range of 0.2-8.0%, asiaticoside in the range of 0.1-11.0% and madecassoside in the range of 0.1-13.0%.

5. The synergistic herbal composition as claimed in claim 1, wherein the magnesium content in the salt or complex of asiatic acid and salt or complex of madecassic acid is in the range of 0.1% to 10%.

6. The synergistic herbal composition as claimed in claim 1, wherein the extract, fraction, active compound, or phytochemical corresponding to each ingredient in the composition is obtained from at least one plant part selected from the group consisting of leaves, stems, tender stems, tender twigs, aerial parts, whole fruit, fruit rind, seed, root, bark, bulb, hardwood, gum resin or whole plant or mixtures thereof.

7. The synergistic herbal composition as claimed in claim 6, wherein the composition is standardized to at least one Centella asiatica marker selected from asiatic acid, madecassic acid, asiaticoside, and madecassoside or total asiaticosides; and to at least one Theobroma cacao phytochemical reference marker compound or pharmacologically active marker but not limited to theobromine.

8. The synergistic herbal composition as claimed in claim 1, wherein the solvent for the extracts or fractions or mixtures thereof is selected from C1-C5 alcohols such as ethanol, methanol, n-propanol, isopropyl alcohol; ketones such as acetone, methylisobutyl ketone, chlorinated solvents such as methylene dichloride and chloroform; C1-C7 hydrocarbons such as hexane, heptane; esters such as ethyl acetate and mixtures thereof; water and mixtures thereof.

9. The synergistic herbal composition as claimed in claim 1, wherein the compositions comprises optionally at least one additional component selected from the group consisting of biological agent(s) and nootropic agent(s); pharmaceutically acceptable active ingredients, vitamins, minerals; pharmaceutically or nutraceutically or dietically acceptable, excipients, carriers or diluents.

10. The synergistic herbal composition as claimed in claim 9, wherein the pharmaceutically or nutraceutically or dietically acceptable excipients, carriers and diluents are selected from monosaccharides such as glucose, dextrose, fructose, galactose etc.; disaccharides such as but not limited to sucrose, maltose, lactose, lactulose, trehalose cellobiose, chitobiose etc.; polycarbohydrates such as starch and modified starch such as sodium starch glycolate, pre-gelatinized starch, soluble starch, ultrasperse A and other modified starches; dextrins that are produced by hydrolysis of starch or glycogen such as yellow dextrin, white dextrin, maltodextrin, glucidex 12D, rice maltodextrin, tapioca maltodextrin etc.; polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide, syloids etc.; metallic stearates such as but not limited to calcium stearate, magnesium stearate, zinc stearate etc.; organic acids such as citric acid, tartaric acid, malic acid, succinic acid, lactic acid, L-ascorbic acid etc.; fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; natural pigments, flavors, Class I & Class II preservatives and aqueous, alcoholic, hydro-alcoholic, organic solutions of above listed ingredients alone or in combination.

11. The synergistic herbal composition as claimed in claim 1, wherein the composition is formulated into a dosage form selected from dry powder form, liquid form, beverage, food product, dietary supplement, or any suitable form such as a tablet, a capsule, a soft chewable tablet, gummies or gummy bar.

12. The synergistic herbal composition as claimed in claim, 1 wherein the composition is formulated into a nutritional/dietary supplement that can be contemplated/made into the dosage form of healthy foods or food for specified health uses such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

13. The synergistic herbal composition as claimed in claim 1, wherein the composition is formulated into a controlled release tablet, using controlled release polymer based coatings by the techniques including nanotechnology, microencapsulation, hot-melt extrusion, colloidal carrier systems, and other drug delivery systems for obtaining the desired therapeutic benefit.

14. A process for preparing a synergistic herbal composition comprising: a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents; which comprises the following steps of:
(a) extracting dried Centella asiatica powder with suitable solvent;
(b) treating the solution of step (a) with a magnesium source;
(c) filtering the solution of step (b) to obtain the first extract and spent Centella asiatica powder;
(d) extracting the spent Centella asiatica powder with a second solvent;
(e) treating the solution of step (d) with a magnesium source;
(f) filtering the solution of step (e) to obtain the second extract;
(g) combining both extracts obtained in steps (c) and (f);
(h) evaporating the solvent of step (g) and drying the residue to obtain Centella asiatica extract blend containing magnesium salt or complex of asiatic acid and madecassic acid along with asiaticoside and madecassoside;
(i) optionally, evaporating the filtrate of step-(c) by omitting steps (d) to (h) and drying the residue to obtain Centella asiatica extract containing magnesium salt or complex of asiatic acid and madecassic acid; along with asiaticoside and madecassoside;
(j) blending of extract of step (h) or step (i) with Theobroma cacao extract in the presence of a suitable solvent; and optionally with suitable excipients; and
(k) drying the product to get the required composition.

15. The process for preparing the synergistic herbal composition as claimed in claim 15, wherein the suitable solvent is selected from but not limited to C1-C5 alcohols, like ethanol, methanol, n-butanol, water, and mixtures thereof, ketones selected from acetone, methylisobutyl ketone, chlorinated solvents selected from methylene dichloride and chloroform; water and mixtures thereof, C1-C7 hydrocarbons such as hexane, heptane; esters such as ethyl acetate and mixtures thereof.

16. The process for preparing the synergistic herbal composition as claimed in claim 15, wherein the magnesium source is selected from the oxides, hydroxides, or carbonates; examples include magnesium oxide, magnesium hydroxide, and magnesium carbonate.

17. A method of obtaining at least one benefit related to cognitive function, mental ability and/or nootropic activity selected from alertness, vigilance, attention, focus/concentration, neuromotor function (reaction time), memory (verbal, working, visual, logical), intelligence, learning, acquisition, retention, recall information/communication, perception, reasoning, problem solving, decision making, and; reducing mental fatigue and improving gaming performance; wherein the method comprises supplementing the human in need thereof with an effective dose of a composition comprising combination of a first ingredient selected from Centella asiatica extract containing at least one component selected from magnesium salt or complex of asiatic acid and magnesium salt or complex of madecassic acid or mixtures thereof along with asiaticoside and madecassoside; and a second ingredient selected from extract, fraction, phytochemical or mixtures thereof derived from Theobroma cacao; further containing optionally at least one component selected from pharmaceutically or nutraceutically or dietically acceptable excipients, carriers and diluents.

18. (canceled)
